(19) United States
(12) Patent Application Publication
(10) Pub. No.: US 2019/0224456 A1
(43) Pub. Date: Jul. 25, 2019

(54) GRADED STIFFNESS GUIDEWIRE

(71) Applicants: University of Maryland, Baltimore, Baltimore, MD (US); University of Maryland Medical System, LLC, Baltimore, MD (US)

(72) Inventors: Zachary Kon, New York, NY (US); Bartley P. Griffith, Gibson Island, MD (US); Mehrdad Ghoreishi, Baltimore, MD (US); Chetan Pasrija, Gaithersburg, MD (US); Gregory Bittle, Baltimore, MD (US)

(21) Appl. No.: 16/256,499

(22) Filed: Jan. 24, 2019

Related U.S. Application Data
(60) Provisional application No. 62/621,174, filed on Jan. 24, 2018.

(51) Int. Cl. A61M 25/09

(52) U.S. Cl. CPC A61M 25/09; A61M 2025/0915; A61M 2025/09133

(57) ABSTRACT

Disclosed herein is a guidewire configured for placement of large bore catheters and cannulae inside of a patient's blood vessels without fluoroscopic guidance. The guidewire, in accordance with certain aspects of an embodiment of the invention, comprises sections of varying stiffness throughout optimally fixed length portions of the guidewire that particularly ease placement of such large bore catheters and cannulae in ECMO applications.

### CROSS REFERENCE TO RELATED APPLICATION

This application claims the benefit of U.S. Provisional Application No. 62/621,174 titled “Graded-Stiffness Wire,” filed Jan. 24, 2018 by the inventors herein, which application is incorporated herein by reference in its entirety.

### FIELD OF THE INVENTION

The present invention relates generally to guidewires used to advance cannuale and catheters into a patient's body, and more particularly to guidewires used for percutaneously placing large bore cannulae or catheters in blood vessels of a patient with or without fluoroscopic guidance.

### BACKGROUND

Guidewires are used in a variety of medical procedures for percutaneous placement of a device, such as a catheter or cannula, into a patient's blood vessel or other body lumen. In various applications, the distal tip of the guidewire is percutenously introduced into the patient's vasculature, and the distal tip is guided under the manipulation of the operator to its desired position within the patient's body. For instance, guidewires may be introduced through a patient's femoral artery for purposes of carrying out a cardiac procedure, such as angioplasty, which advances an angioplasty catheter over the guidewire to the site requiring angioplasty treatment.

Of course, tactile feedback to the operator is important during such processes to ensure that excessive force is not applied that might damage the patient's vasculature. Likewise, providing the tip with a sufficiently flexible configuration is important so as to prevent damage to the patient's vasculature. As applications such as angioplasty typically use relatively small diameter catheters, maintaining appropriate tactile feedback while minimizing risk of damage from the distal end of the guidewire has, to at least some extent, been addressed by prior known, small diameter (e.g., approximately 0.011 to about 0.017 inch diameter) guidewires.

However, certain medical procedures require use of large bore catheters (and particularly catheters sized 14 gauge or “14G” to 16 gauge or “16G”), such as extracorporeal membrane oxygenation (“ECMO”), which use such large bore catheters that supply extracorporeal cardiac and respiratory support to patients whose lungs are unable to provide an adequate amount of gas exchange or perfusion to sustain life. Placement of the large bore cannulas or catheters necessary to carry out ECMO is significantly more difficult to carry out without damaging the patient's vasculature, as the 14G and 16G cannulae and catheters are more rigid and can easily displace a small and more flexible guidewire that would typically be used for advancing, for instance, and angioplasty catheter. However, simply increasing the overall size and/or stiffness of previously known guidewires to account for the increased stiffness of 14G and 16G cannulae creates highly increased risk of damage to the patient's vasculature as the guidewire is advanced to its intended location.

Thus, there remains a need in the art for a guidewire that has sufficient stiffness to safely, percutaneously introduce a large bore catheter into a patient's artery, but that is otherwise configured to minimize risk of damage to the patient's vasculature as it is advanced to the intended location of the distal end of such large bore catheter.

### SUMMARY OF THE INVENTION

Disclosed herein is a guidewire configured for placement of large bore catheters and cannulae inside of a patient's blood vessels without fluoroscopic guidance. The guidewire, in accordance with certain aspects of an embodiment of the invention, comprises sections of varying stiffness throughout optimally fixed length portions of the guidewire that particularly ease placement of such large bore catheters and cannulae in, for example, ECMO applications.

In accordance with certain aspects of an embodiment of the invention, a guidewire having a distal end and a proximal end opposite the distal end is provided, the guidewire comprising: a distal portion having a distal portion first end coinciding with the distal end of the guidewire, and a distal portion second end opposite the first end, the distal portion extending 5 cm-18 cm from the distal end and having a flexural modulus of between 0.1 GPa and 5 GPa; an intermediate portion having an intermediate portion first end coinciding with the distal portion second end and an intermediate portion second end opposite the first end, the intermediate portion having a length of 5 cm-30 cm from the intermediate portion first end to the intermediate portion second end and having a flexural modulus of between 5 GPa and 20 GPa; and a proximal portion having a proximal portion first end coinciding with the distal portion second end, the proximal portion having a flexural modulus of between 40 GPa and 70 GPa.

In accordance with further aspects of an embodiment of the invention, a method for inserting a large bore catheter or cannula into a patient's blood vessel is provided, comprising: providing a guidewire having a distal end and a proximal end opposite the distal end, the guidewire comprising: a distal portion having a distal portion first end coinciding with the distal end of the guidewire, and a distal portion second end opposite the first end, the distal portion extending 5 cm-18 cm from the distal end and having a flexural modulus of between 0.1 GPa and 5 GPa; an intermediate portion having an intermediate portion first end coinciding with the distal portion second end and an intermediate portion second end opposite the first end, the intermediate portion having a length of 5 cm-30 cm from the intermediate portion first end to the intermediate portion second end and having a flexural modulus of between 5 GPa and 20 GPa; and a proximal portion having a proximal portion first end coinciding with the distal portion second end, the proximal portion having a flexural modulus of between 40 GPa and 70 GPa; and inserting a catheter or cannula having a size of 14G to 16G over said proximal portion of the guidewire at a point of insertion of said catheter or cannula into a patient's blood vessel.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 shows a schematic, side sectional view of a guidewire having portions of differing stiffness created through use of differing materials in accordance with certain aspects of an embodiment of the invention.

FIG. 2 shows a schematic, side sectional view of a guidewire having portions of differing stiffness created through use of varying thicknesses of wire and outer material in accordance with further aspects of an embodiment of the invention.

FIG. 3 shows a schematic, side sectional view of a guidewire having portions of differing stiffness created through use of a continuously tapering thickness of wire in accordance with still further aspects of an embodiment of the invention.

FIG. 4 shows a schematic, side sectional view of a guidewire having portions of differing stiffness created through use of stiffening elements in accordance with still further aspects of an embodiment of the invention.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

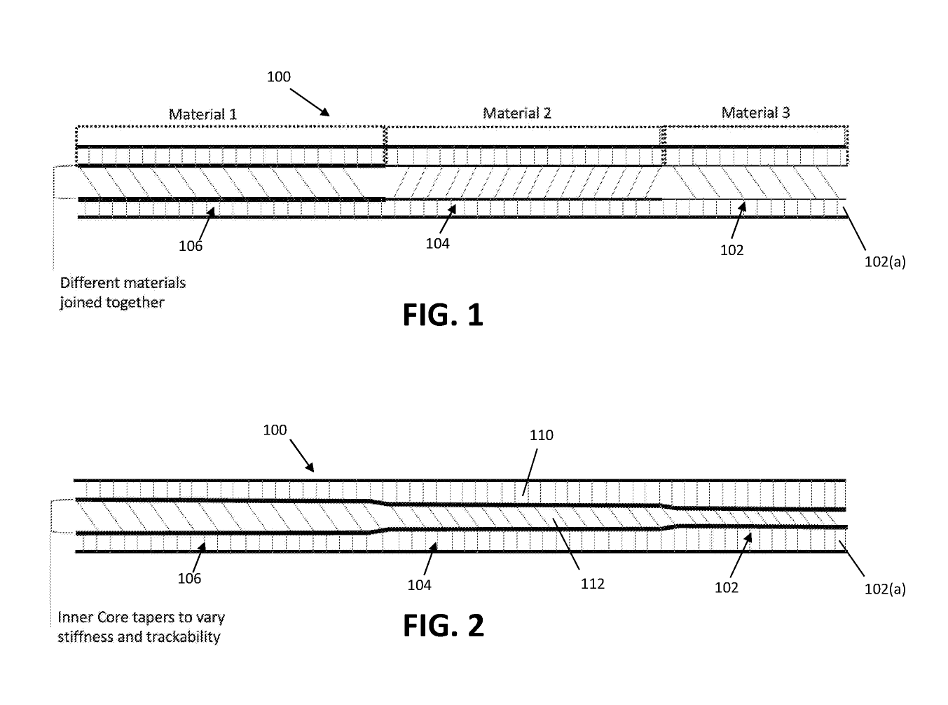

With reference to FIGS. 1-4, a guidewire apparatus 100 is provided for percutaneous placement of large bore catheters or cannulae (e.g., 14G to 16G) into a patient's blood vessel with or without fluoroscopic guidance. In accordance with certain aspects of an embodiment, a guidewire 100 configured as described herein may be used for placement of an extracorporeal membrane oxygenation cannula. The guidewire has a graded stiffness along its length, and more particularly specific, varying stiffnesses along specific lengths of the guidewire, which specific stiffnesses and lengths have been found by the inventors herein to be optimal for percutaneous placement of large bore catheters and cannulas, such as those used in extracorporeal membrane oxygenation. The guidewire includes a distal portion that may be considered “floppy” or less stiff than the other portions of the guidewire, an intermediate portion that has a stiffness that is greater than the stiffness of the distal portion, and a proximal portion having a maximum stiffness that is greater than the stiffness of each of the distal portion and the intermediate portion.

A guidewire 100 configured in accordance with at least certain aspects of an embodiment increases the ease of guiding a catheter, and more particularly a large bore catheter, into a patient's vessel along a tract as compared to typical wires. In accordance with certain aspects of an embodiment, the stiffness of a guidewire 100 at the point of entry into the patient's blood vessel is high so as to effectively guide the distal tip 102(a) of the large bore catheter or cannula into the patient's blood vessel without injury. Likewise, the stiffness of the distal tip of the guidewire is low in order to increase trackability and decrease the risk of puncturing vessels or organs as the guidewire is advanced. An intermediate segment 104 of graded stiffness is interposed between the distal, flexible tip 102(a) of the guidewire 100 and the stiff body of the proximal portion 106 of the guidewire 100 in order to increase the bend radius of the leading end of the guidewire, which prevents the stiff portion 106 from overtaking the flexible, distal tip 102(a), and acts as a spring to modulate force transmission to the distal tip 102(a) and provides increased haptic feedback to the operator. These characteristics allow the guidewire 106 formed in accordance with certain aspects of an embodiment to be passed at bedside, optionally without fluoroscopic assistance, which is conventionally used when passing stiff guidewires in order to decrease the risk of causing injury with a more stiff distal guidewire tip. Thus, the guidewire 100 according to certain embodiments combines the benefits of typical stiff wires and typical floppy wires while eliminating the need for fluoroscopic guidance.

The specific length and total diameter of a guidewire 100 formed in accordance with an embodiment herein may vary depending upon the specific intended application, and more particularly the access location, the vessel intended to receive the large bore catheter or cannula, and the particular procedure (e.g., ECMO) that is to be carried out. In a particularly preferred embodiment, such a guidewire has a total diameter of 0.035″. Likewise in such particularly preferred embodiment, such a guidewire has a total length between 120-180 centimeters.

In applications in which such a guidewire is to be used for femoral venous or arterial access, the guidewire 100 may have an exemplary total length of approximately 180 centimeters, and a total diameter of approximately 0.035″. In this configuration, the guidewire 100 includes a distal portion 102 that extends proximally between 8-18 centimeters from the distal tip 102(a) of the guidewire, with such distal portion 102 being generally flexible, and more particularly more flexible than the other proximal portions of the guidewire. For example, the distal portion 102 may be configured to allow for atraumatic entry into a patient's vessel, such that the distal portion 102 has a stiffness and trackability similar to a typical Benston wire. The distal portion 102 of the guidewire 100 in such configuration generally exerts an axial force of approximately between 0.1 N and 1 N when navigating through a patient's vessel, and exerts a bending force of approximately between 0.5 N and 2 N. Further, the distal portion 102 may have a flexural modulus of approximately between 0.1 GPa and 5 GPa.

Proximal to such distal portion 102 on a guidewire 100 configured for femoral access is an intermediate portion 104, having a length preferably of approximately between 10 cm-30 cm, and more preferably between 15 cm-25 cm, immediately adjacent to the distal portion 102. Such intermediate portion 104 is configured to have a stiffness and trackability that are greater than the distal portion 102 (as described above) and less than the proximal portion 106 (as described below). The intermediate portion 104 itself may be graded in stiffness along the length of the intermediate portion 104, such that the distal end of the intermediate portion 104 has a stiffness and trackability similar to a typical Bentson wire, while the proximal end of the intermediate portion 104 has the characteristics of the proximal portion 106 of the guidewire. In an exemplary configuration, the average stiffness and trackability of the intermediate portion 104 may be similar to the stiffness and trackability of a typical Amplatz wire, but as noted above may vary along the length of the intermediate portion 104. The intermediate portion 104 of the guidewire generally exerts an axial force of approximately between 0.5 N and 1.5 N when navigating through a patient's vessel, and exerts a bending force of approximately between 0.5 N and 1.5 N. Further, the intermediate portion 104 may have a flexural modulus of approximately between 5 GPa and 20 GPa.

Guidewires are generally inserted into the patient's vessel such that the following catheter or cannula follows the course of the guidewire. Thus, the guidewire is always beyond the tip of the catheter until the catheter is safely in position and the guidewire is removed. Large bore cannulae, such as those used for ECMO, are typically inserted between 10 cm-60 cm. With the foregoing guidewire configuration inserted into the femoral vein or artery, the guidewire 100 can be inserted such that the proximal, stiff portion 106 of the guidewire is at the point of vessel entry, while the distal, flexible portion 102 of the guidewire is safely distal to the tip of the catheter or cannula. Moreover, with a guidewire 100 configured as above, there is not an excessive amount of wire inserted into the patient's vessel before reaching the proximal, stiff segment 106. This is important, as even with a flexible guidewire, excessive wire insertion can result in vessel injury or touching the patient's heart, potentially resulting in arrhythmia.

Proximal to such intermediate portion 104 on a guidewire 100 configured for femoral access is the proximal portion 106, which extends from the proximal end of the intermediate portion 104 to the proximal end of the guidewire outside of the patient. Optionally, the proximal portion 106 as referred to herein need not comprise the full length of the guidewire between the intermediate portion 104 and the free, proximal end, and could instead comprise only a length of guidewire, e.g., 30 cm-35 cm, immediately proximal to the intermediate portion 104, with the remainder of the guidewire having any configuration suited to the particular operation. The proximal portion 106 of the guidewire 100 is configured to place large bore catheters or cannulae into a patient's vessel in a safer operation than is afforded by previously known guidewire methods and devices. For example, the proximal portion 106 of the guidewire 100 is stiffer than the intermediate portion 104 and the distal portion 102 so as to act as a rail for placement of large bore catheters or cannulae. As a further example, the proximal portion 106 has a stiffness and trackability that is similar to an Amplatz SUPERSTIFF wire. In a particular embodiment, the proximal portion 106 exerts a bending force of approximately between 1 N and 3 N. Further, the proximal portion 106 may have a flexural modulus of approximately between 40 GPa and 70 GPa.

Similarly, in applications in which such a guidewire 100 is to be used for jugular venous access, the guidewire may have an exemplary total length of approximately 120 cm, and a total diameter of approximately 0.035″. In this configuration, the distal portion 102 of the guidewire 100 may have a length of approximately between 5 cm-15 cm that is flexible (as described above). Further, the intermediate portion 104 of such a guidewire 100 may have a length of approximately between 5 cm-15 cm (proximal to the distal portion) and a stiffness and trackability as described above with respect to the intermediate portion 104 of the femoral configuration. Still further, the proximal portion 106 of the guidewire (proximal to the intermediate portion) may again extend proximally from the intermediate portion 104, and may have a stiffness and trackability similar to an Amplatz SUPERSTIFF wire (and as described above with respect to the proximal portion 106 of the femoral configuration).

A guidewire 100 configured as described above provides multiple degrees of stiffness and trackability along its length, such as along the distal portion 102, intermediate portion 104, and proximal portion 106 of the guidewire 100, in specific stiffness values and located at specific regions of the guidewire that are optimal for placement of large bore catheters and cannulae. Such varying guidewire stiffnesses may be provided by a variety of varying mechanical devices and material treatment methods.

For example, and as shown in the schematic view of a guidewire 100 of FIG. 1, a guidewire 106 configured as described above may be manufactured from a plurality of differing materials, which may include by way of non-limiting example stainless steel, Nitinol and other shape memory alloys, and similarly configured bio-compatible materials having varying stiffnesses as detailed above. In this configuration, those distinct materials provide each of the distal portion 102, intermediate portion 104, and proximal portion 106 a differing stiffness and trackability, all as described above. Each portion is axially coupled, such as (by way of non-limiting example) through welding end portions of each segment together to form the full length of the guidewire 100 having the total diameter of each portion. The stiffness and trackability of each portion may further vary according to typical methodologies, such as by forming each portion from different materials having different stiffnesses and trackabilities. For example, each portion may be formed of different metallic or material compositions, heat treatments, surface treatments, and the like, to have different stiffnesses and trackabilities.

Similarly, FIG. 2 is a schematic view of a guidewire 100 configured as described above and that is manufactured from a distal portion 102, an intermediate portion 104, and a proximal portion 106, each having a particular cross-section. For example, each portion may have a solid circular-like cross-section or a cross-section having an aperture. The aperture of each portion is preferably generally located centrally in the cross-section, and may have an elliptical-like shape, although other shapes such as triangular, rectangular, and other shapes are feasible without departing from the invention. In certain configurations, the aperture may include a separate and distinct material from the guidewire itself. Thus, each portion may have a different stiffness and trackability, all as described above. Each portion is axially coupled (e.g., the distal portion 102 is coupled to the intermediate portion 104, which in turn is coupled to the proximal portion 106) to form the length of the guidewire 106 having the total diameter of each portion.

Optionally, and with continued reference to FIG. 2, each portion of the guidewire 100 may have a different sub-diameter that is encased in an outer material 110. Generally, the outer material 110 for each portion has a thickness that is the difference between the total diameter of the guidewire 100 and the sub-diameter of the interior wire 112 of each portion. The outer material 110 may be formed of a variety of typical biocompatible materials, such as coatings, metals, plastics, rubbers, and the like. Thus, each portion of the guidewire 100 may have a different stiffness and trackability as described above, according to the total diameter, the outer material 110, and the outer material thickness. Each portion is axially coupled (e.g., the distal portion 102 is coupled to the intermediate portion 104, which in turn is coupled to the proximal portion 106) to form the length of the guidewire having the total diameter of each portion.

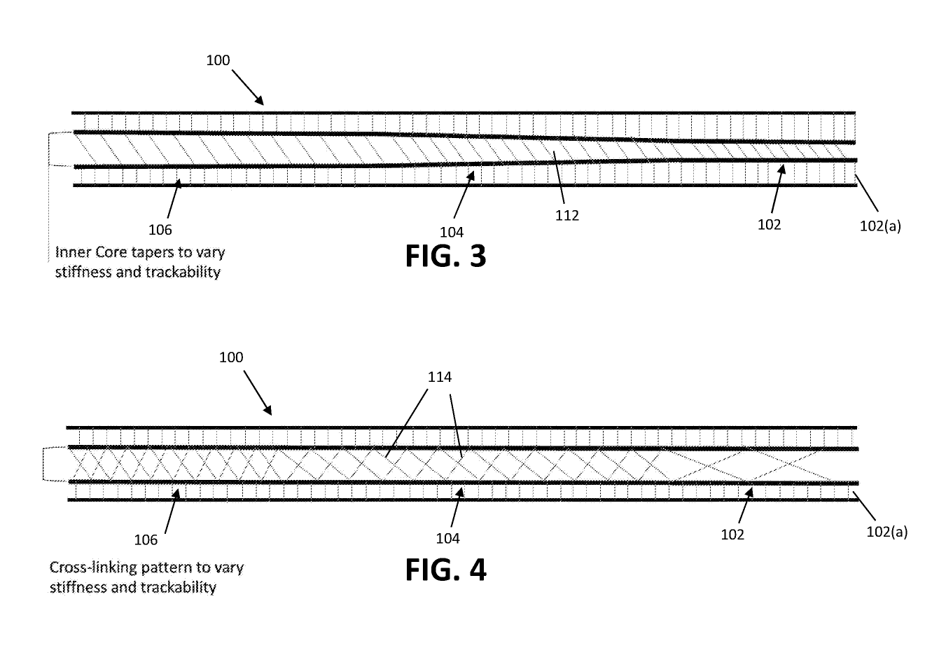

Further, and with particular reference to FIG. 3, the intermediate portion 104 may comprise a continuously tapering portion of internal wire 112 that narrows in diameter from the end of intermediate portion 104 adjacent to the proximal portion 106 to the opposite end of the intermediate portion 104 adjacent to the distal portion 102, with the stiffness of the intermediate portion 104 continuously decreasing from the proximal end to the distal end. The smooth taper of the intermediate portion 104 in such configuration allows the guidewire to curve smoothly and have more spring without kinking along its length (e.g., where the stiffness abruptly changes), thus reducing the risk of damage to the patient's blood vessel while still allowing optimal control for the physician. The wire 112 in such configuration may be formed by taking a core wire having a diameter of the proximal (i.e., stiffest and largest diameter) portion 106 and grinding it along its length to form a continuously tapering intermediate portion 104, after which the distal end of the tapered intermediate portion 104 may be bonded to a flexible distal portion 102 of the guidewire. While the foregoing thus envisages forming such guidewire 100 by bonding two pieces, the guidewire 100 may likewise be formed by bonding and grinding, or optionally drawing, different combinations of core portions, thus forming the guidewire 100 with continuously tapering portions of either one, two, or three separate core portions.

Even further, FIG. 4 is a schematic view of a guidewire 100 configured as described above and in which each of the distal portion 102, intermediate portion 104, and proximal portion 106 includes stiffening elements 114 to affect the stiffness and trackability of each such portion as detailed above. For example, stiffening elements 114 may comprise helical wires that wrap each portion of the guidewire 100, wherein the helical wires have many properties, including a pitch angle, cross-section, and thickness. The pitch angle (i.e., the angle of each helical wire relative to an axis of each portion of the guidewire 100) is generally between 0 and 90 degrees, such that a portion of a guidewire 100 having helical wires with higher pitch angles may generally be stiffer than a portion of a guidewire 100 having helical wires with lower pitch angles. Further, each portion of the guidewire 100 may have many helical wires, wherein each helical wire may have different pitch angles. Still further, some helical wires may have negative pitch angles (e.g., cross-linking wires). The helical wires may be formed of many materials, such as metals, plastics, or composites (e.g., glass fibers) according to the desired stiffness for each respective portion detailed above.

Optionally, a guidewire 100 formed in accordance with aspects of an embodiment may include a combination of the configurations discussed above and shown in FIGS. 1-4 to form a guidewire 100 having portions of different stiffnesses, such as a distal portion 102 that is floppy (i.e., more flexible than the remaining portions of the guidewire), an intermediate portion 104 that has a higher stiffness than the distal portion 102, and a proximal portion 106 that has a higher stiffness than the intermediate portion 104. In certain exemplary configurations, a guidewire 100 formed in accordance with aspects of an embodiment may include an outer coating (e.g., rubberized, heparinized) according to the desired application of the guidewire 100. In still further exemplary configurations, the stiffness and trackability of each portion of a guidewire 100 formed as discussed herein may vary substantially continuously along each portion of the guidewire.

In each of the foregoing configurations, the guidewire 100 may also include external markings or indicia indicating the depth of insertion of the guidewire into the patient's vessel. Such markings may include, by way of non-limiting example, continuous segments of distinct colors or patterns demarcating the segments of varying stiffness, continuous segments of distinct colors or patterns indicating actual depth of insertion, and/or discreet markings spaced at regular intervals (e.g., hash marks every 10 cm). Such markings allow the operator, in the absence of fluoroscopic guidance, to ensure that the guidewire 100 has been placed at a depth that is sufficient to guide the catheter or cannula that is being placed, but not at an excessive depth which might cause vessel or organ injury.

Thus, provided herein is a guidewire apparatus particularly configured for percutaneous placement of large bore catheters or cannulae, including an extracorporeal membrane oxygenation cannula. The guidewire includes multiple degrees of stiffness along its length, such as a distal portion that is most flexible, an intermediate portion that is more stiff than the distal portion, and a proximal portion that is more stiff than the intermediate portion. A guidewire configured as detailed above may increase the ease of guiding a catheter or cannula, and more particularly a large bore catheter or cannula, into a patient's vessel along a tract without fluoroscopic assistance.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A guidewire having a distal end and a proximal end opposite said distal end, said guidewire comprising:
a distal portion having a distal portion first end coinciding with said distal end of said guidewire, and a distal portion second end opposite said first end, said distal portion extending 5 cm-18 cm from said distal end and having a flexural modulus of between 0.1 GPa and 5 GPa;
an intermediate portion having an intermediate portion first end coinciding with said distal portion second end and an intermediate portion second end opposite said first end, said intermediate portion having a length of 5 cm-30 cm from said intermediate portion first end to said intermediate portion second end and having a flexural modulus of between 5 GPa and 20 GPa; and
a proximal portion having a proximal portion first end coinciding with said distal portion second end, said proximal portion having a flexural modulus of between 40 GPa and 70 GPa.

2. The guidewire of claim 1, wherein said intermediate portion has a continuously decreasing stiffness along said length of said intermediate portion from said intermediate portion second end to said intermediate portion first end.

3. The guidewire of claim 1, said distal portion extending 8 cm-18 cm from said distal end.

4. The guidewire of claim 1, said distal portion extending 5 cm-15 cm from said distal end.

5. The guidewire of claim 1, said intermediate portion having a length of 10 cm-30 cm from said intermediate portion first end to said intermediate portion second end.

6. The guidewire of claim 1, said intermediate portion having a length of 15 cm-20 cm from said intermediate portion first end to said intermediate portion second end.

7. The guidewire of claim 1, said intermediate portion having a length of 5 cm-15 cm from said intermediate portion first end to said intermediate portion second end.

8. The guidewire of claim 1, further comprising indicia on an outer surface of said guidewire configured to indicate a depth of insertion of the guidewire into a patient's blood vessel.

9. A method for inserting a large bore catheter or cannula into a patient's blood vessel, comprising:
providing a guidewire having a distal end and a proximal end opposite said distal end, said guidewire comprising:
a distal portion having a distal portion first end coinciding with said distal end of said guidewire, and a distal portion second end opposite said first end, said distal portion extending 5 cm-18 cm from said distal end and having a flexural modulus of between 0.1 GPa and 5 GPa;
an intermediate portion having an intermediate portion first end coinciding with said distal portion second end and an intermediate portion second end opposite said first end, said intermediate portion having a length of 5 cm-30 cm from said intermediate portion first end to said intermediate portion second end and having a flexural modulus of between 5 GPa and 20 GPa; and
a proximal portion having a proximal portion first end coinciding with said distal portion second end, said proximal portion having a flexural modulus of between 40 GPa and 70 GPa; and
inserting a catheter or cannula having a size of 14G to 16G over said proximal portion of said guidewire at a point of insertion of said catheter or cannula into a patient's blood vessel.

10. The method of claim 9, wherein said step of inserting a catheter or cannula further comprises inserting said catheter or cannula into a patient's femoral blood vessel.

11. The method of claim 10, wherein said distal portion extends 8 cm-18 cm from said distal end.

12. The method of claim 10, wherein said intermediate portion has a length of 10 cm-30 cm from said intermediate portion first end to said intermediate portion second end.

13. The method of claim 10, wherein said intermediate portion has a length of 15 cm-20 cm from said intermediate portion first end to said intermediate portion second end.

14. The method of claim 9, wherein said step of inserting a catheter or cannula further comprises inserting said catheter or cannula into a patient's jugular blood vessel.

15. The method of claim 14, wherein said distal portion extending 5 cm-15 cm from said distal end.

16. The method of claim 14, wherein said intermediate portion has a length of 5 cm-15 cm from said intermediate portion first end to said intermediate portion second end.

17. The method of claim 9, wherein said guidewire further comprises indicia on an outer surface of said guidewire configured to indicate a depth of insertion of the guidewire into a patient's blood vessel.
